(19) United States
(12) Patent Application Publication
(10) Pub. No.: US 2022/0005569 A1
(43) Pub. Date: Jan. 6, 2022

(54) SYSTEM AND METHOD TO AUTOMATICALLY RECOMMEND AND ADAPT A TREATMENT REGIME FOR PATIENTS

(71) Applicant: PREDICTMEDIX INC., Toronto (CA)

(72) Inventor: Rajiv Muradia, Ottawa (CA)

(21) Appl. No.: 17/384,686

(22) Filed: Jul. 23, 2021

Related U.S. Application Data
(60) Provisional application No. 63/048,131, filed on Jul. 5, 2020.
(60) Provisional application No. 63/048,152, filed on Jul. 5, 2020.
(60) Provisional application No. 63/058,567, filed on Jul. 30, 2020.

(51) Int. Cl. G16H 20/00; G16H 10/60; G06N 20/00

(52) U.S. Cl. CPC G16H 20/00; G06N 20/00; G16H 10/60

(57) ABSTRACT

A system and method of automatically recommending and adopting the treatment regime for a patient suffering from a disease is disclosed. The method collects medical data of the patient from one or more medical devices and stores the medical data in a medical database. A logic designer module is utilized for designing the logic for automatic recommendation and adaption of the treatment regime of the patient. Machine learning techniques are used to further refine the automatic recommendation and adaption of the treatment regime of the patient. A clinical controller and scheduler module schedules the recommended treatment regime for the patient. The scheduled recommendation and adaption regime can be displayed in real time to a clinical dashboard and mechanisms are provided for interaction with the patient for information dissemination and feedback.

### RELATED APPLICATIONS

This application is related to, and claims priority to, the following:
1. Provisional Application Ser. No. 63/048,131, filed Jul. 5, 2020.
2. Provisional Application Ser. No. 63/048,152, filed Jul. 5, 2020.
3. Provisional Application Ser. No. 63/058,567, filed Jul. 30, 2020.

The subject matter of the related applications, each in its entirety, is expressly incorporated herein.

### FIELD OF THE INVENTION

The present invention relates to methods and systems of care management to automatically recommend and adapt to a treatment regimen for a patient.

### BACKGROUND OF THE INVENTION

The present invention provides a method and system of management to automatically recommend and adapt to a treatment regimen. More specifically, the systems and methods for automatic recommendation and treatment regime for a patient may involve gathering patient data, patient inputs, and treatments recommended by a treatment pathway controller to identify time series event, apply rules for diagnosis of disease, requesting or receiving an approval from a medical expert, and providing the detailed treatment plan for the patient.

A patient may be critically ill or may not be in a condition to visit the hospital or a medical physician on regular basis. Such restriction may result in further deterioration of health of the patient. For example, a patient may be bed ridden but otherwise perfectly in good health but may require necessary monitoring of the health condition. At the same time, the patient may also be reluctant to go to the hospital. In another scenario, the patient may be in a critical condition and may need a life-saving treatment even before the patient may reach the hospital. All these scenarios require a system that may automatically recommend and adapt to a treatment so that both, the lifestyle management and/or prescriptive analytics, may provide better health management to the patient.

To overcome and better manage a chronic disease, a platform is needed that utilizes the patient history, clinical data, DNA profile and captures real time medical data such as blood pressure or blood glucose level to provide prescriptive analytics for chronic disease management and in some critical cases life saving management by providing time to the patient to reach the hospital. The prescriptive analytics provides intelligent recommendations for the next step of optimal care pathways to drive positive health outcomes for the patient.

### SUMMARY OF THE INVENTION

A system and method of automatically recommending and adopting the treatment regime for a patient suffering from a disease is disclosed. The method collects medical data of the patient from one or more medical devices and stores the medical data in a medical database. A logic designer nodule is utilized for designing the logic for automatic recommendation and adaption of the treatment regime for the patient. The logic designer module passes the logic designer data to a machine learning module. The machine learning module also receives the medical data of the patient from the medical database. The machine learning module then creates the automatic recommendation and adaption regime based on the logic designer configured by the medical expert using the clinical user interface and the medical data from the medical database. A clinical controller and scheduler module schedules the automatic recommendation and adaption regime for the patient. A clinical dashboard module displays in real time the scheduled recommendations and adaption regime to a clinical dashboard. The scheduling and adaption of treatment and feedback for compliance of the automatic recommendation and adaption of treatment regime is displayed on a patient device.

In another embodiment, system and method that provides a prescriptive analytics platform for managing the care path of the patient is disclosed. The system includes a data collector module that captures all type of data including real time medical data of the patient from the patient's personal device. The medical data and other related disease data collected from the data aggregator module is passed onto a data filtering module that segments the data into different categories and uses the specific categorized data for machine learning and for formulation of a treatment plan. In yet another embodiment, the data collector may aggregate data from external sources such as health insurance companies, hospital, family doctor etc. and internal sources such as real time data of patient monitoring at home, such as drug response, speech data, mood, feeling, anxiety and clinical parameters such as body temperature, body weight, blood pressure and other possible parameters related to medical data. In another embodiment, the filtered data may be passed to a transformation module to transform and/or reduce and/or normalize the data for prescriptive analysis, big data analytics, reporting, machine learning or for some other transformative prescriptive analytics. The transformed data may be utilized for automated or manual learning of the automatic recommendation and adaption system. In some embodiments, the machine learning module creates the automatic recommendation and adaption regime based on the logic designer module and logic data created by a medical expert using the clinical user interface. A clinical controller and scheduler module may schedule the automatic recommendation and adaption regime for the patient. A clinical dashboard module displays in real time the scheduled recommendation and adaption regime to a dashboard. In some embodiments, the clinical dashboard module may display data in one or more user interfaces of the patient device, the wellness management partner's interface and the disease management partner's interface. In some embodiments, the clinical dashboard module may display data on the patient device and the wellness or care pathway interface device. The scheduling and adaption of treatment and feedback for compliance of the automatic recommendation and adaption of treatment regime is displayed on a patient device.

In some embodiments, the scheduling and adaption of treatment may be controlled by the machine learning module automatically.

In some embodiments, the scheduling and adaption of treatment may be controlled by the learning algorithms based on decision tree module and/or rule based engine.

In some embodiments, the scheduling and adaption of treatment may be controlled by the learning algorithms based on a rule based engine. The rules may be defined by the disease management partner or by a medical expert.

In some embodiments, the type of data collected by the data aggregator module may include medical history of the patient, current and past clinical test data, hereditary data, DNA profile, and real time data that is captured by one or more medical devices at different instances of time in a specified period, for example, a day, a week, a month and the like.

In some embodiments, the data filtering may be performed based on disease type, for example, chronic disease management, event prediction such as occurrence of a heart stroke, prediction of other diseases, or readmission to a hospital after discharge or some other type of health prediction.

### DESCRIPTION OF FIGURES

Different embodiments will now be described in detail with reference to the drawings, in which:

FIG. 1 illustrates the overall environment n block form of an automatic recommendation and adaption system in accordance with an embodiment of the present invention;

FIG. 2 illustrates the different blocks of the automatic recommendation and adaption system in an embodiment of the present invention;

FIG. 3 is a process flow chart of the automatic recommendation and adaption system in an embodiment of the present invention;

FIG. 4 illustrates an exemplary user interface of the automatic recommendation and adaption system, which includes condition templates and condition template workbench in an embodiment of the invention;

FIG. 5 illustrates an administrative template interface of the automatic recommendation and adaption system in an embodiment of the invention;

FIG. 6 illustrates a patient care interface of the automatic recommendation and adaption system an embodiment of the invention;

FIG. 7 illustrates the different blocks of the automatic recommendation and adaption system along with a reward management system in an embodiment of the present invention.

### DETAILED DESCRIPTION OF THE INVENTION

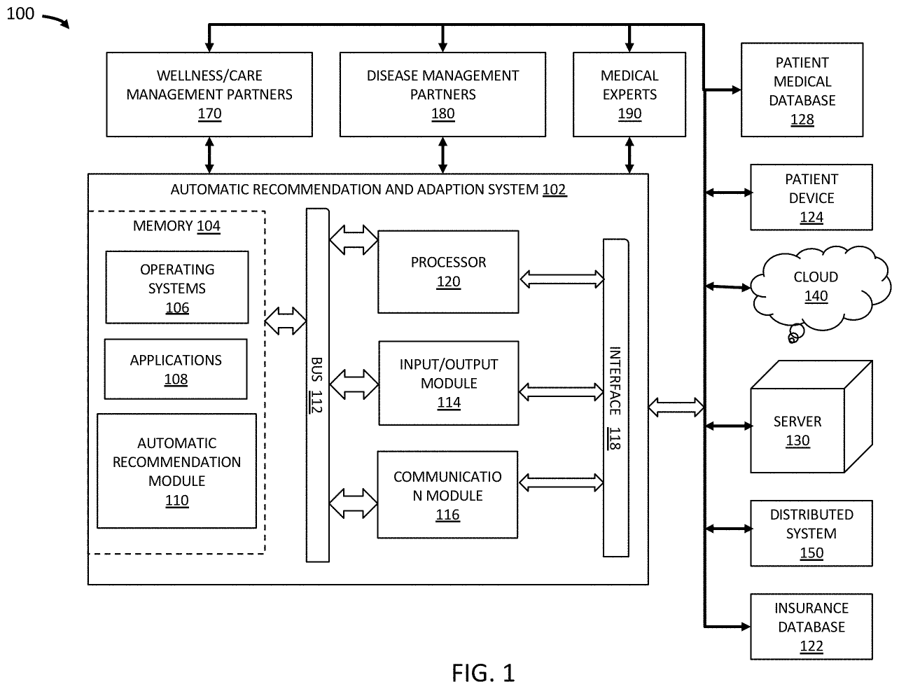

FIG. 1 illustrates the overall environment 100 in which an automatic recommendation and adaption system operates for providing care pathway to a patient. The overall environment 100 includes the automatic recommendation and adaption system 102, one or more wellness management partners 170 (also referred as care management partner), one or more disease management partners 180, one or more medical experts 190, a data patient medical database 128, a patient device 124, a cloud 140, a server 130, a distributed system 150 and an insurance database 122.

In some embodiments, one or more wellness management partners 170 may be implemented as a software module or process in the server 130 or the distributed system 150 and connected with the automatic recommendation and adaption system 102.

In some embodiments, one or more disease management partners 180 may be implemented as a software module or process on the server 130 or the distributed system 150 and connected with the automatic recommendation and adaption system 102.

In some embodiments, the one or more medical experts 190 may be implemented as a software module or process on the server 130 or the distributed system 150 and connected with the automatic recommendation and adaption system 102.

The automatic recommendation and adaption system 102 includes a memory 104 comprising an operating system 106, one or more applications 108, and an automatic recommendation module 110. The automatic recommendation module 110 is embedded in the memory 104 of the automatic recommendation and adaption system 102 in the exemplary embodiment of FIG. 1, however, in other embodiments, the automatic recommendation module 110 may be provided in a server 130, a cloud 140 or a distributed system 150. The automatic recommendation and adaption system 102 further includes a processor 120, an input/output module 114, and a communication module 116, an internal bus 112 and an external interface 118. The internal bus 112 allows transfer of data between the memory 104 and the processor 120, the input/output module 114, and the communication module 116. Additionally, the external interface 118 allows the automatic recommendation and adaption system 102 to communicate and exchange data with the cloud 140, the server 130, and the distributed system 150. Furthermore, the interface 118 allows communication with wellness management partner 170, the disease management partner 180, the medical experts 190, the patient device 124, the insurance database 122, and the patient medical database 128.

The one or more wellness management partner 170 may be a hospital, a medical agency, a clinic or a medical laboratory that manages the treatment and care path of the patient. In some embodiments, wellness management partner 170 may be more than one, each having access to the automatic recommendation and adaption system 102. The one or more wellness management partner 170 may submit, access, edit, modify, and delete information in automatic recommendation and adaption system 102 through a user interface.

In some embodiments, the one or more wellness partner may be a care management partner. In some embodiments, the one or more wellness partner may also be the one or more care management partner and may be used interchangeably.

The one or more disease management partner 180 may be a medical research institute, a hospital, a clinic or a medical laboratory that specializes in chronic disease management of one or more chronic disease. The one or more disease management partner 180 may submit, access, edit, modify, and delete information in automatic recommendation and adaption system 102 through a user interface.

In some embodiments, the one or more medical experts 190 may be a panel of experts that provide education guidance to the patients through directed research on one or more chronic disease. The one or more medical experts 190 may submit, access, edit, modify, and delete information n automatic recommendation and adaption system 102 through a user interface.

The patient device 124 may be any type of handheld computing device such as, but not limited to, a mobile phone, PDA, tablet, medical device with a user interface. In some embodiments, the patient device 124 may be implemented as a software program, which nay be installed on a computer, a laptop, a desktop or some other type of computing device. In some embodiments, the patient device may be a medical instrument having a user interface having wired or wireless access to the automatic recommendation and adaption system 102.

In some embodiments, the patient device 124 may a medical application that may be available through an online app store such as stores for android and iOS devices which is capable of interfacing with the automatic recommendation and adaption system 102.

The insurance database 122 may contain information related to the patient and the insurance history related to disease and events that occurred in life history of the patient including, but not limited to, chronic disease flair ups, the frequency of hospitalization and other parameters related to the history of the patient. In some embodiments, insurance plans may be directly linked for availing the incentives based on absence of insurance claims for the patient.

In some embodiments, the patient medical database 128 may comprise one or more interfaces for collection of patient data from physicians, doctors, hospital, clinics, nursing homes, pathology labs, DNA labs, government medical database or some other database that may hold patient data or information. All the medical data or information related to the patient may be aggregated in the patient medical database 128. In different embodiments, the patient medical database 128 may be implemented as a standalone database, a distributed database, a flat file system or another type of database.

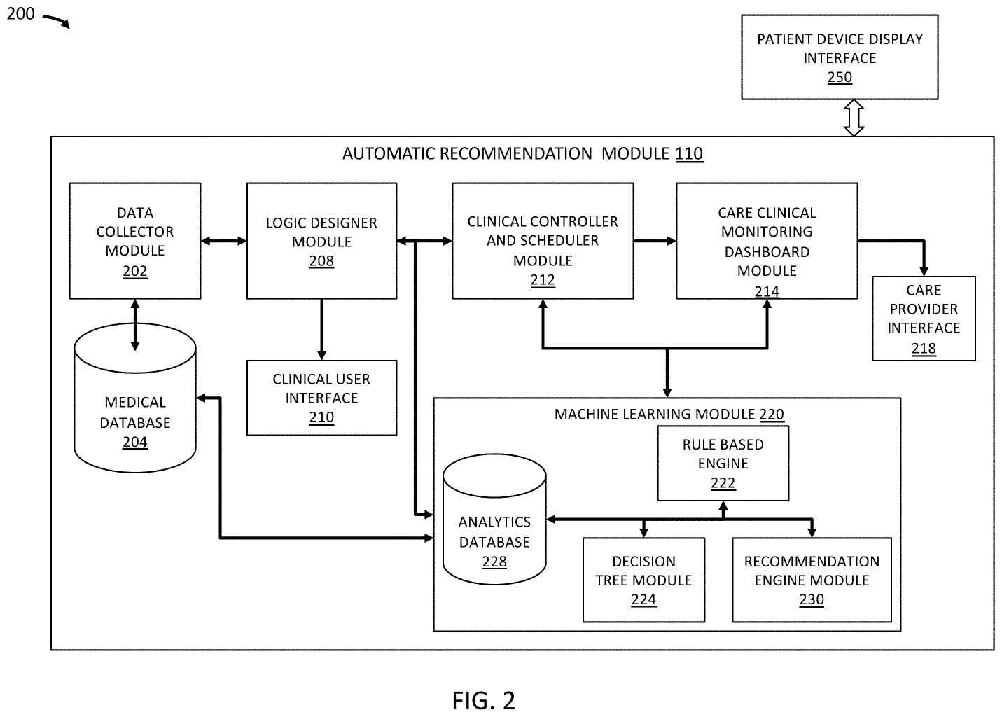

FIG. 2 illustrates the various components of the automatic recommendation module 110 in an embodiment of the present invention. The automatic recommendation module 110 comprises of a data collector module 202, a medical database 204, a logic design module 208, a clinical user interface 210, a clinical controller and scheduler module 212, a care clinical monitoring dashboard module 214, a care provider interface 218 and a machine learning module 220.

In some embodiments, the data collector module 202 may include a memory and a processor.

In some embodiments, the logic designer module 208 may include a memory and a processor.

In some embodiments, the clinical user interface 210 may include a memory and a processor.

In some embodiments, the clinical controller and scheduler module 212 may include a memory and a processor.

In some embodiments, the care clinical monitoring dashboard module 214 may include a memory and a processor.

In some embodiments, the machine learning module 220 may include a memory and a processor.

In some embodiments, the data collector module 202 may be integrated with one or more medical devices and/or the patient device 124 for capturing real time data of the patient. In some embodiments, the data collector module 202 may be integrated with a patient device display interface 250 having a display interface to show care pathway to the patient. In some embodiments, the data collector module 202 is integrated with the patient medical database 128 for accessing medical history of the patient for a care pathway or a treatment plan.

In some embodiments, the logic designer module 208 may allow one or more of the administrators, user, medical experts, disease management partners, and care management partners to create, edit, change, modify or delete the templates. The template may be in an XML format or some other format.

The logic designer module 208 allows the administrators, user, medical experts, disease management partners, and care management partners to dynamically update existing template with new or updated rules and to insert, modify, create or delete disease level definition rules. In an embodiment, the logic designer 208 may implement drools flow designer.

In some embodiments, the patient device display interface 250 may be implemented with dedicated hardware and in other embodiments as a software module on a mobile device of the patient. In some embodiments, the mobile device of the patient may be a medical device 124. In some embodiments, the mobile device may be a mobile phone, a PDA and other type of mobile device having software application to interface with the automatic recommendation and adaption system 102.

The clinical controller and scheduler module 212 may check the validity of the logic designer module 208, ensure that the result from logic designer module 208 is complete before sending it to the patient. It may also resolve any conflict between changes to the the logic designer module 208 from clinical expert, care provider and/or the patient.

The care clinical monitoring dashboard module 214 may provide status of existing care pathways for the patient. Furthermore, the clinical monitoring dashboard module 214 may provide recommendations on next steps based on consent.

The care provider interface 218 is a patient interface for self management. In some embodiments, the patient may delegate the care provider interface 218 to a third party, such as but not limited to, nurse, a care management agency, a coach or some other person for management and compliance feedback to the automatic recommendation module 110.

The machine learning module 220 comprises of a rule based engine 222, an analytics database 228, a decision tree module 224, and a recommendation engine module 230. The rule based engine 222 stores rules that are created/modified/stored for each care pathway. In embodiments, the rule based engine 222 may be implemented with drool guvnor. The analytics database 228 includes the rules for automated recommendations. The decision tree module 224 enables decisions based on rules applied to or arrived at by consideration of the feedback data being received from the patient for their specific care pathways. The recommendation engine module 230 provide recommendations based on results from rules execution that may be viewed and overridden by the patient (in self-care mode) and the care provider (in assisted mode).

In various embodiments, the machine learning module 220 may include techniques based on nearest neighbor, k-nearest neighbors, support vector machines, naive Bayesian, decision trees, random forests, logistic regression, and/or linear discriminate analysis. In some embodiments, the machine learning module may implement artificial neural networks, deep learning, support vector machines and others.

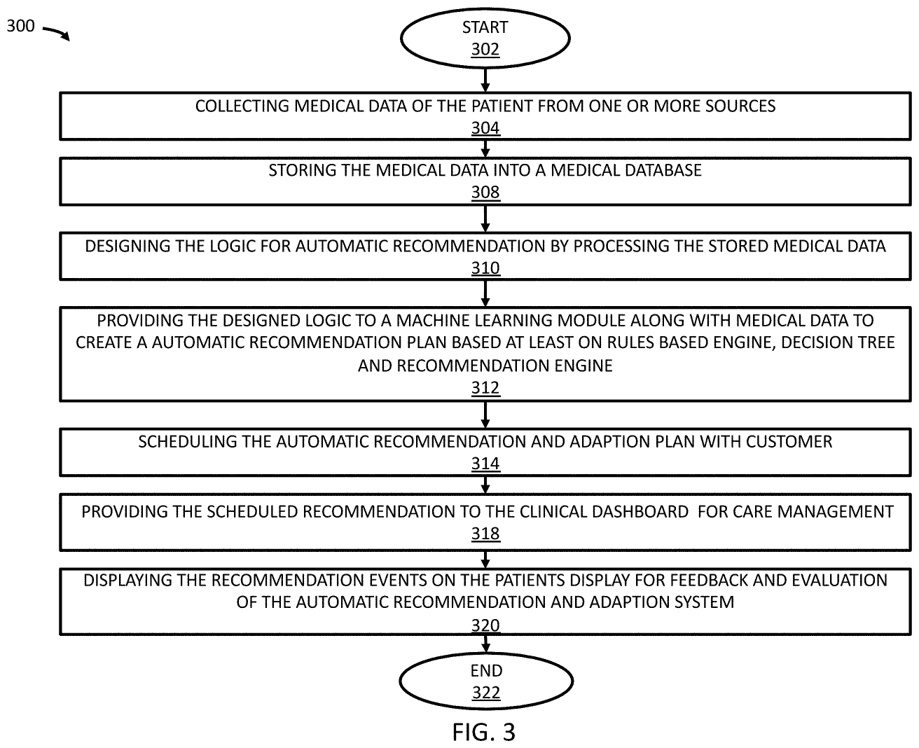

FIG. 3 illustrates the process flow for automatic recommendation and adaption method of treatment regime for a patient. The process 300 is initiated at step 302 and immediately moves to a step 304. At step 304, the process 300 collects the medical data for the patient from one or more sources. In some embodiments, the one or more sources may be patient device 124 or the patient device display interface, the patient medical database 128, the insurance database 122 and other sources of aggregating medical data of the patient. At step 308, the collected medical data of the patient is stored in the database 204. The medical data stored in the medical database 204 may be provided to machine learning module 220 and the logic designer module 208. At step 310, the medical expert may create a disease template or use an existing disease template for creating a design logic for the patient treatment and recommendation based at least on the medical data of the patient and other parameters associated with the patient. In some embodiments, the disease management partner may create a logic design for treatment and recommendation. In some embodiments, the one or more disease management partners may create individual templates of the logic design for treatment and recommendation and submit the template for evaluation and suggestion of the medical expert and/or patient. The patient may provide feedback as to how much time the patient may devote to automatic recommendation and treatment regime suggested by the medical expert. In some embodiments, the suggestion given by the patient may be considered by the machine learning module 220 to provide customization of the disease template according to patient requirements.

In some other embodiment, the disease management partner may get in touch with the patient to design logic and treatment plan. At step 312, the design logic is passed to the machine learning module 220 and the clinical controller and scheduler module 212. The machine learning module 220 includes the analytics database 228 connected with the rule engine module 222, the decision tree module 224 and the recommendation engine module 230 to create an automatic recommendation plan based on the design logic and the medical data of the patient. In some embodiments, the analytics database may proactively analyze the patient data using the decision algorithm and deep learning to predict preventive health care issues in context of the ongoing disease management. For example, the likelihood of a hypertension patient developing diabetes due to hereditary reasons.

In some embodiments, the machine learning algorithm 220 may use decision tree module 224 and recommendation module 230 to predict the likelihood of a stroke in future based on current medical data of the patients. Numerous other predictions and preventive treatments may be forecasted by the automatic recommendation a module 110; all these embodiments fall within the scope of the present invention.

In some embodiments, the logic designer module 208 is connected with the machine learning module 220, which develops a care management plan and provides it to the clinical controller and scheduler module 212. In some embodiments, the machine learning module 220 develops a care management plan and monitors the adaption plan automatically using artificial intelligence. In some embodiments, the machine learning module 220 develops a care management plan and monitors the adaption plan automatically, which is supervised by the care management partner.

At step 314, the process 300 receives the automatic recommendation and adaption plan for the patient from the machine learning module 220 and the clinical controller and scheduler module 212 passes the recommendation plan to the patient for implementation and adoption. In some embodiments, the automatic recommendation and adaption plan for the patient received from the machine learning module 220 is reviews, altered or modified by a care management partner. In some embodiments, the automatic recommendation and adaption plan for the patient is implemented after review by the disease management partner.

Moving further at step 318, the automatic recommendation schedule and adaption is provided on the clinical monitoring dashboard module 214 of the care management partner for adaption and compliance monitoring.

In some embodiments, the automatic recommendation schedule and adaption is provided on the patient device 250 for self monitoring and compliance.

In some embodiments, the automatic recommendation schedule and adaption is provided on the clinical monitoring dashboard module 214 of the care management partner for adaption and compliance monitoring.

In some embodiments, the automatic recommendation schedule and adaption is implemented and monitored by the machine learning module 220 by directly communicating with the patient device 250.

At step 320, the recommendation, adaption, non-compliance, compliance reward data and other parameters are directly displayed on the patient device 250. The process 300 may continue iteratively until the prescribed medical health condition is achieved or unless terminated by the administrator. At step 322, the process 300 terminates.

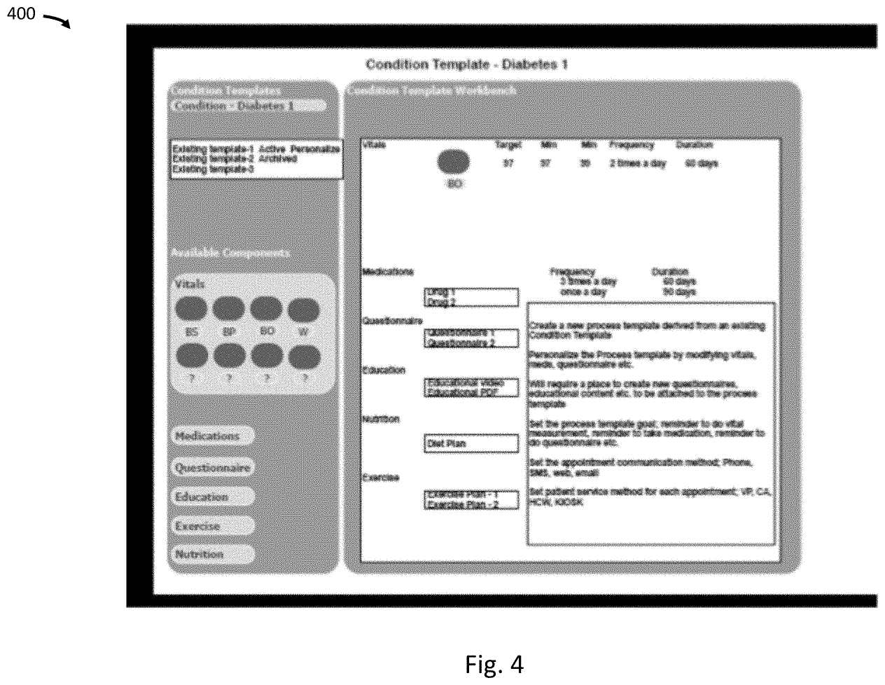

FIG. 4 illustrates a user interface on the care provider interface in an embodiment of the present invention. In another embodiment, the user interface 400 may be provided to one or more disease management partners 180. In yet another embodiment, the user interface 400 may be provided to one or more wellness/care management partners 170.

As illustrated in user interface 400, the automatic recommendation and adaption system 102 includes condition templates and condition template workbench. The condition templates are structured data formats of available information related to the patient. Likewise, the condition template workbench allows to access, edit, modify and implement the target plan for the patient. In this embodiment, an exemplary example of the type I diabetes is illustrated, however, in other embodiments other chronic disease such as but not limited to CHF (including hypertension, heart attack), COPD, arthritis, obesity, stroke, stress, anxiety may be treated. The user interface provides access to all the information that has been aggregated or stored or accessible to the automatic recommendation and adaption system 102. In an exemplary illustration, the user interface 400 provides different templates for a specific chronic condition. For example, the current template is for active type 1 diabetes. Other templates may be activated on the user interface by a drop-down menu. The user interface 400 shows the active vitals, for example blood pressure reading over time by selecting the BP button. Additionally, all the information related to the patient may be displayed by pressing the related buttons. For example, the medication button, questionnaire button, nutrition button and other buttons as shown.

In embodiments, the medical information related to patient may be medication details, questionnaire filed by patients from time to time, education related to chronic disease, exercise schedule of present and past, and nutrition plans for past and present or some other type of medical information related to patient history and treatment. The condition template workbench allows you to access the stored information such as medication plan, nutrition plan, education plan, and exercise plan shared with the patient. In addition, the condition template workbench allows the administrator/medical expert to edit the information to formulate a new plan for the patient.

Finally, the user interface shows the prescribed treatment plan for the patient with the target value, minimum value, frequency and duration for the patient for the current disease care plan.

In some embodiments, the administrator/medical expert may look into the prescribed treatment plan for the patient with the target value, minimum value, frequency and duration for the patient for the previous plans.

In some embodiments, the administrator/medical expert may look into the questionnaires filled by the patient.

In some embodiments, the administrator/medical expert may look into the previous treatment plan for the patient.

In some embodiments, the administrator/medical expert may look into the previous exercise plan for the patient.

In some embodiments, the automatic recommendation and adaption system may be displayed on the patient device, which connects with the automatic recommendation and adaption system by wired or wireless connection.

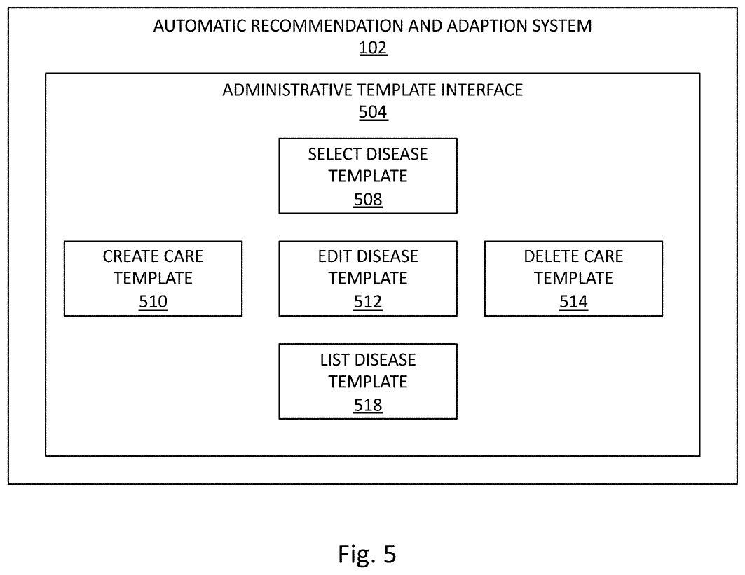

FIG. 5 illustrates the administrative template interface of the automatic recommendation and adaption system 102 in an embodiment of the present invention. The administrative template interface 504 includes a UI comprising buttons, drop-down menu, scroll bar, selection bar and other UI components to create, select, modify and delete templates for automatic recommendation of patient care pathway. The administrative template interface 504 comprises a select disease template 508 to identify a disease, for example, a chronic disease such as diabetes and to create a template for automatic and/or manual management of the disease by activating the select disease template button 508. In some embodiments, the select disease template 508 may be selected either by the medical expert or a disease management partner or a care management partner.

The administrative template interface 504 includes a create care template 510 to create a care template for a disease, for example, a chronic disease such as diabetes for automatic and/or manual management of the disease. In some embodiments, the create care template 508 may be created either by the medical expert or a disease a management partner or a care a management partner.

The administrative template interface 504 further includes an edit disease template 512 to edit the contents of the disease or to add or delete certain parameters. In some embodiments, the parameters associated with edit diseases template 512 may be treatment method, lifestyle changes, corrective measure, medicine and other parameters related to automatic disease management and adaption.

The administrative template interface 504 further includes a delete care template 514 to delete care template for management of a disease. In addition, the administrative template interface 504 includes a list disease template that allows the user/care administrator/medical expert to list and review the stored disease templates.

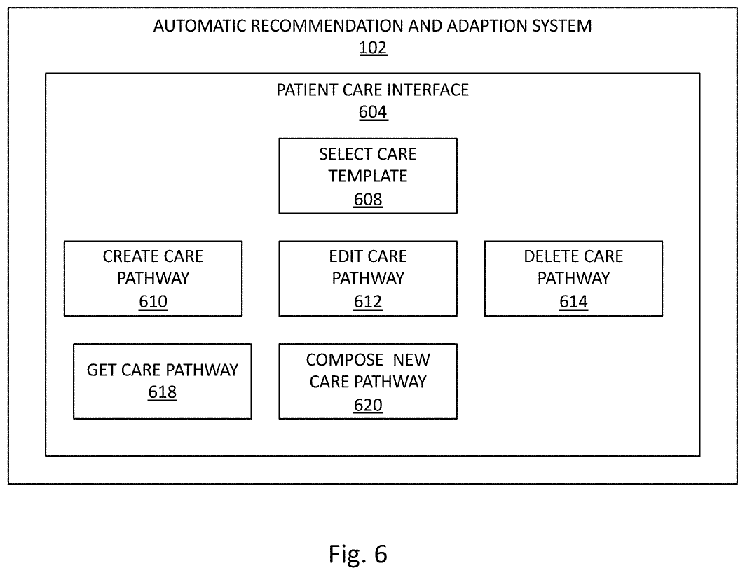

FIG. 6 illustrates the patient care interface of the automatic recommendation and adaption system in an embodiment of the present invention. The patient care interface 604 includes a user interface, which comprises buttons, drop-down menu, scroll bar, selection bar and other user interface components to create, select, modify and delete templates for automatic recommendation of patient care pathway. The patient care interface 604 comprises a select care template 608. The select care template allows the care partner to select appropriate care template for a disease management associated with the patient. For example, a chronic disease such as diabetes may have different templates for type I, type II, and type III diabetes. In some embodiments, the select care template 608 may only be initiated by the care management partner.

Further, the patient care interface 604 includes a create care pathway 610, an edit care pathway 612, a delete care pathway 614, a get care pathway 618, and a compose new care pathway 620.

The create care pathway 610 allows the care management partner to prepare a roadmap for management of the disease of their patients, which may include daily routines, medication, and exercises. In some embodiments, multiple care pathway may be prepared with at least one active care pathway for automatic recommendation and adaption. In some embodiments, existence of multiple care pathway may imply that the patient has one or more disease that require care and treatment.

The edit care pathway 512 may allow the care management partner to make correction to automatic care treatment of the patient. In some embodiments, the correction may be related to medicine, lifestyle management, exercise or correction required after a discovery of new scientific data for disease management.

The delete care pathway 614 allows one or more care management partner to delete a plan for care pathway for a specific patient.

The get care pathway 618 allows one or more care management partner to retrieve a plan for care pathway for a specific patient.

The compose new care pathway 620 allows one or more care management partner to formulate a new plan for care pathway for a specific patient. In some embodiments, the new care pathway plan may be related to discovery of a new medicine, lifestyle management, new exercise plan or correction required after a discovery new scientific data for disease management. Alternatively, the new care pathway plan may be used to create a new pathway plan due to patient's health improving and patient being requiring less drastic care pathway after following the previous recommended plan.

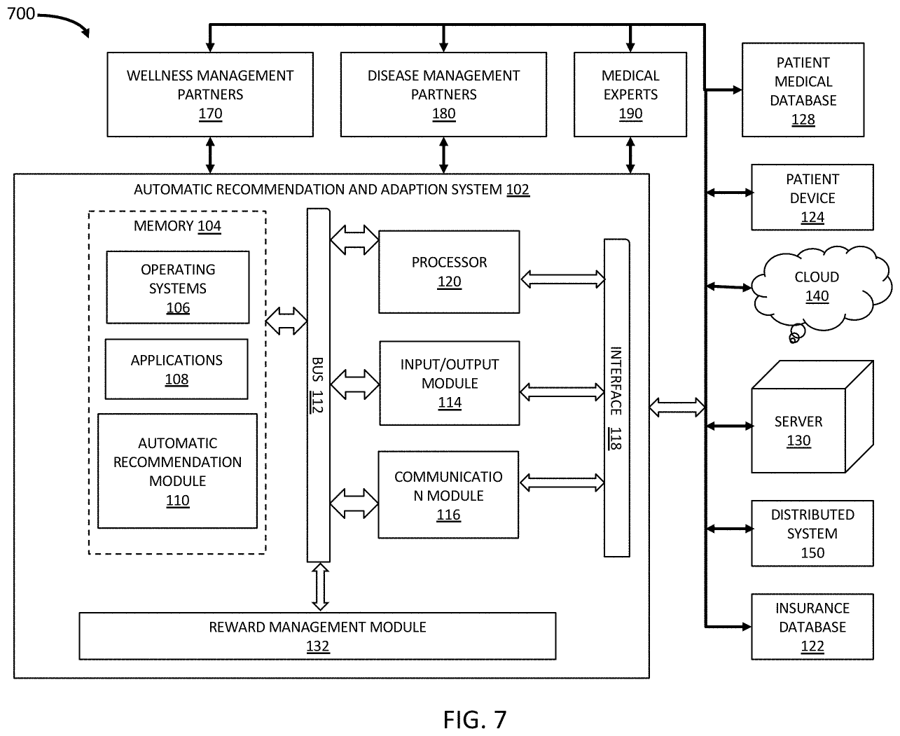

FIG. 7 illustrates the integration of the automatic recommendation and adaption system 102 with a rewards system by integrating an additional rewards management system 132 in an embodiment of the present invention whereas the rewards system may be utilized to encourage compliance with the care pathway. The reward management module 132 integrated with different components of the automatic recommendation and adaption system 102 allows the automatic recommendation and reward management systems to be implemented in one system.

In some embodiments, the automatic recommendation and adaption system 102 may be integrated externally with a reward management module 132. Alternatively, the reward management module 132 may be implemented as part of the automatic recommendation and adaption system 102 as shown. The reward management method and system provide an interface to remotely access, monitor and reward a patient/user for compliance to a prescribed medical treatment by providing reward points. The reward points may be converted into coupons, offers or promissory notes that may be used or redeemed by the patient/user for goods and services. The method collects the medical data of the patient/user using one or more medical devices. The captured medical information/data may be converted into events based on the medical data and/or user attributes. The events may be transformed with a set of rules stored in a normalized configuration database into normalized reward events. The normalized reward events may then be transformed into reward points by implementing the rules embedded in the normalized reward rules database. The reward events are as a result converted into normalized reward points. The normalized reward points are stored into reward database. In addition, the normalized reward points are converted into coupons, offers or promissory notes, which may be used or redeemed for tangible goods and/or services.

Disclosed is a method of automatically recommending and adapting the treatment regime for a patient suffering from a disease. The method comprises (a) collecting a medical data of the patient from one or more medical devices, (b) storing one or more medical data in a medical database, (c) designing the logic for automatic recommendation and adaption of the treatment regime of the patient, (d) providing the logic designer to a machine learning processor along with the medical data of the patient to create the automatic recommendation and adaption regime, (e) scheduling and monitoring the automatic recommendation and adaption regime for the patient, (f) displaying in real time the scheduled recommendation and adaption regime to a clinical dashboard, and monitoring and evaluating the real time feedback from the patient related to the automatic recommendation and adaption regime.

Disclosed is an automatic recommendation and adaption system for treatment regime of a patient suffering from a disease, the automatic recommendation and adaption system includes a data collector for collecting medical data of the patient from one or more medical devices. A medical database for storing medical data in a medical database and a logic designer module for designing the logic for automatic recommendation and adaption of the treatment regime of the patient. Further, the automatic recommendation and adaption system includes a machine learning processor configured to access the medical database to create the automatic recommendation and adaption regime for treatment and a clinical controller and scheduler module for scheduling and monitoring the automatic recommendation and adaption regime for the patient. In addition, it includes a clinical monitoring dashboard module for displaying in real time the scheduled recommendation and adaption regime to a clinical dashboard, and a care provider interface for monitoring and evaluating the real time feedback from the patient related to the automatic recommendation and adaption regime.

In some embodiments, at least one or more medical devices may comprise a patient device having a display for viewing real time dashboard related to compliance with automatic treatment regime of the patient.

In some embodiments, the medical database may be connected to a patient medical database having life history of the patient. Further, the medical database may be connected to an insurance database.

In some embodiments, the process of designing the automatic recommendation and adaption may be performed by a logic designer configured to allow one or more disease management partners for creating a disease template.

In some embodiments, the machine learning processor may comprise an analytics database, a rule based engine, a recommendation engine, and a decision tree.

In some embodiments, the analytics database may store the rules for the automatic recommendation and adaption regime. In some embodiments, the automatic recommendation and adaption regime may be managed by the machine learning processor with manual intervention. In some embodiments, the automatic recommendation and adaption regime may be managed by a care management partner. In some embodiments, the automatic recommendation and adaption regime may be displayed on the patient dashboard. In some embodiments, the automatic recommendation and adaption regime may be displayed on the care management partner for monitoring compliance and adaption assessment.

We claim:

1. A method of automatically recommending and adapting a treatment regime for a patient suffering from a disease, the method comprising:
collecting medical data of the patient from a medical device;
storing the medical data in a medical database;
designing the logic for an automatic recommendation and adaption of the treatment regime of the patient by using a logic designer;
providing the logic designer results to a machine learning processor along with the medical data of the patient to create the automatic recommendation and adaption regime;
scheduling and monitoring the automatic recommendation and adaption regime for the patient;
displaying in real time the scheduled recommendation and adaption regime to a clinical dashboard, and
monitoring and evaluating the real time feedback from the patient related to the automatic recommendation and adaption regime.

2. The method of claim 1, wherein the medical device comprises a patient device having a display for viewing at least one of a real time dashboard and the automatic recommendation and adaption regime.

3. The method of claim 1, wherein the medical database is connected to a patient medical database with a life history of the patient.

4. The method of claim 1, wherein the medical database is connected to an insurance database.

5. The method of claim 1, wherein the logic designer is configured for a disease management partner to create a disease template.

6. The method of claim 1, wherein the machine learning processor comprises an analytics database, a rule based engine, a recommendation engine, and a decision tree.

7. The method of claim 1, wherein the automatic recommendation and adaption regime is managed by the machine learning processor.

8. The method of claim 1, wherein the automatic recommendation and adaption regime is managed by a care management partner.

9. The method of claim 1, wherein the automatic recommendation and adaption regime is displayed on a care management partner dashboard for compliance and adaption assessment.

10. The method of claim 6, wherein the analytics database stores a set of rules for the automatic recommendation and adaption regime.

11. An automatic recommendation and adaption system for a treatment regime of a patient suffering from a disease, the automatic recommendation and adaption system comprising:
a data collector for collecting a medical data of the patient from a medical device;
a medical database for storing the medical data;
a logic designer module for designing a logic for the automatic recommendation and adaption of the treatment regime of the patient;
a machine learning processor configured to use the medical database to create the automatic recommendation and adaption regime for treatment;
a clinical controller and scheduler module configured to schedule and monitor the automatic recommendation and adaption regime for the patient;
a clinical monitoring dashboard module configured to display in real time the scheduled automatic recommendation and adaption regime to a clinical dashboard, and
a care provider interface configured to monitor and evaluate the real time feedback from the patient related to the automatic recommendation and adaption regime.

12. The automatic recommendation and adaption system of claim 11, further comprising a patient device with a display for viewing at least one of a real time dashboard and the automatic recommendation and adaption regime for treatment.

13. The automatic recommendation and adaption system of claim 11, wherein the medical database is connected to a patient medical database with a life history of the patient.

14. The automatic recommendation and adaption system of claim 1, wherein the medical database is connected to an insurance database.

15. The automatic recommendation and adaption system of claim 11, wherein the logic designer module is adapted to a clinical user interface for creating a disease template.

16. The automatic recommendation and adaption system of claim 11, wherein the machine learning processor comprises an analytics database, a rule based engine, a recommendation engine, and a decision tree.

17. The automatic recommendation and adaption system of claim 11, wherein the automatic recommendation and adaption regime is managed by the machine learning processor.

18. The automatic recommendation and adaption system of claim 11, wherein the automatic recommendation and adaption regime is configured to be managed by a care management partner.

19. The automatic recommendation and adaption system of claim 11 configured to be displayed on a care management partner display for compliance and adaption assessment.

20. The automatic recommendation and adaption system of claim 16, wherein the analytics database stores the rules for the adapted treatment regime of the patient.
